(19) United States
(12) Patent Application Publication
(10) Pub. No.: US 2016/0206606 A1
(43) Pub. Date: Jul. 21, 2016

(54) TOPICAL COMPOSITIONS COMPRISING A THIAZOLIDINEDIONE

(71) Applicant: Topokine Therapeutics, Inc., Newton, MA (US)

(72) Inventors: Michael S. Singer, Newton Center, MA (US); Murat V. Kalayoglu, Silver Spring, MD (US)

(73) Assignee: Topokine Therapeutics, Inc., Newton, MA (US)

(21) Appl. No.: 15/002,099

(22) Filed: Jan. 20, 2016

Related U.S. Application Data
(63) Continuation of application No. PCT/US2015/031353, filed on May 18, 2015.
(60) Provisional application No. 62/000,932, filed on May 20, 2014.

(51) Int. Cl. A61K 31/4439; A61K 31/427; A61K 9/00; A61K 47/12; A61K 47/10

(52) U.S. Cl. CPC A61K 31/4439; A61K 47/12; A61K 47/10; A61K 9/0014; A61K 31/427

(57) ABSTRACT

Provided herein are compositions comprising a thiazolidinedione compound or pharmaceutically acceptable salt thereof, an alcohol (e.g., a monohydroxy alcohol), and oleic acid, for topical administration to the skin. The thiazolidinedione is advantageously soluble in this vehicle. Further provided are methods for the making the compositions and methods of treatment comprising applying the compositions to the skin of a subject in need thereof.

### RELATED APPLICATIONS

The present application claims priority under 35 U.S.C. §120 to and is a continuation of international PCT Application, PCT/US2015/031353, filed May 18, 2015, which claims priority under 35 U.S.C. §119(e) to U.S. provisional patent application, U.S. Ser. No. 62/000,932, filed May 20, 2014, each of which is incorporated herein by reference.

### FIELD OF THE INVENTION

Provided herein are compositions comprising a thiazolidinedione compound (TZD) or pharmaceutically acceptable salt thereof, for topical administration to the skin. The compositions comprise a TZD, e.g., rosiglitazone, pioglitazone, troglitazone, ciglitazone, netoglitazone, rivoglitazone, or a pharmaceutically acceptable salt thereof, in a vehicle comprising oleic acid and one or more alcohols (e.g., a monohydroxy alcohol such as ethanol or isopropanol). The TZD is surprisingly soluble in this oleic acid/alcohol vehicle, which confers advantages as disclosed herein.

### BACKGROUND OF THE INVENTION

Thiazolidinediones (TZDs), e.g., rosiglitazone, pioglitazone, troglitazone, ciglitazone, netoglitazone, rivoglitazone, and/or pharmaceutically acceptable salts thereof, are a class of structurally related compounds originally developed as antidiabetic agents. These compounds can also be used to treat a dermatologic disease or ameliorate a cosmetic condition in a subject. See, e.g., U.S. Publication 20090042909. For dermatologic or cosmetic use, it is preferable to apply the TZD to the skin, i.e., topically. For topical administration, the TZD is usually combined with a vehicle, e.g., an alcohol-based or water-based vehicle, to obtain a suitable topical composition. Preferably, the TZD is soluble in the vehicle, which promotes uniformity and physical stability in the final formulation. A high degree of solubility (e.g., more than 0.25 mg/mL or more than 4 mg/mL; e.g., more than 0.3 mg/g or more than 4.8 mg/g; e.g., more than 1 part TZD per 200 parts vehicle; or, e.g., more than 1 part TZD per 3000 parts vehicle) may be required because topical compositions deliver drugs by passive diffusion and therefore employ relatively high drug concentrations.

Many topical compositions use a water-based or alcohol-based vehicle; however, TZDs are notoriously insoluble in water and poorly soluble in ethanol. For example, the solubilities of rosiglitazone maleate and pioglitazone hydrochloride in ethanol are about 0.25 and 4 mg/mL, respectively. Thus, it is difficult to make water-based or alcohol-based formulations of TZDs, particularly if the desired final concentration of the TZD is more than 0.25 mg/mL for rosiglitazone maleate, or more than 4 mg/mL for pioglitazone hydrochloride.

Therefore, there is a need for new topical compositions comprising a TZD pharmaceutically acceptable salt thereof.

### SUMMARY OF THE INVENTION

The present invention arises from the discovery that addition of oleic acid to an alcohol-based vehicle substantially and unexpectedly increases the solubility of a TZD compound in the vehicle. Thus, in one aspect, provided is a composition comprising a TZD or pharmaceutically acceptable salt thereof, and a vehicle comprising oleic acid and an alcohol (e.g., a monohydroxy alcohol).

In certain embodiments, the TZD is a compound of Formula (I):

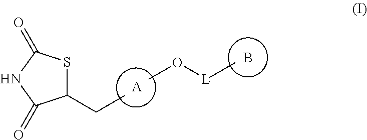

or a pharmaceutically acceptable salt or prodrug thereof;
wherein:

Ring A is substituted or unsubstituted arylene or substituted or unsubstituted heteroarylene;

L is substituted or unsubstituted C₁₋₆alkylene or substituted or unsubstituted heteroC₁₋₆alkylene; and

Ring B is substituted or unsubstituted carbocyclyl, substituted or unsubstituted heterocyclyl, substituted or unsubstituted aryl, or substituted or unsubstituted heteroaryl.

In certain embodiments, the TZD is selected from the group consisting of:

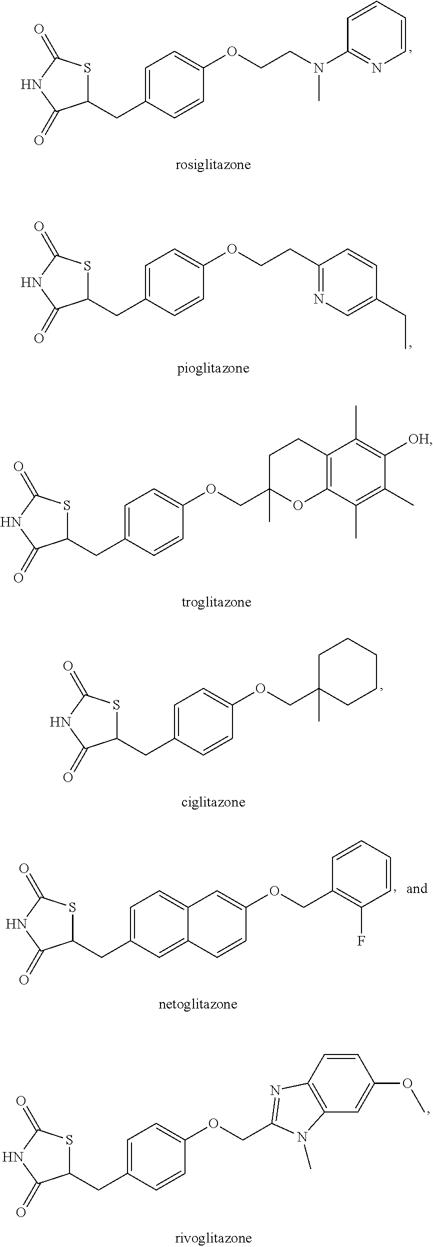 [SMILES: CN(CCOc1ccc(CC2SC(=O)NC2=O)cc1)c1ccccn1 ; CCc1ccc(CCOc2ccc(CC3SC(=O)NC3=O)cc2)nc1 ; Cc1c(C)c2c(c(C)c1O)CCC(C)(COc1ccc(CC3SC(=O)NC3=O)cc1)O2 ; CC1(COc2ccc(CC3SC(=O)NC3=O)cc2)CCCCC1 ; O=C1NC(=O)C(Cc2ccc3cc(OCc4ccccc4F)ccc3c2)S1 ; COc1ccc2nc(COc3ccc(CC4SC(=O)NC4=O)cc3)n(C)c2c1]

and pharmaceutically acceptable salts thereof

In certain embodiments, the TZD or pharmaceutically acceptable salt thereof is rosiglitazone or a pharmaceutically acceptable salt thereof (e.g., rosiglitazone maleate; potassium salt of rosiglitazone). In certain embodiments, the TZD or pharmaceutically acceptable salt thereof is pioglitazone or a pharmaceutically acceptable salt thereof (e.g., pioglitazone hydrochloride; potassium salt of pioglitazone). In certain embodiments, the TZD or pharmaceutically acceptable salt thereof is troglitazone or a pharmaceutically acceptable salt thereof.

In certain embodiments, the concentration of the TZD or pharmaceutically acceptable salt thereof is between about 0.03 percent and about 5 percent by weight, inclusive (i.e., between about 0.3 mg/g and 50 mg/g), e.g., about 0.03 percent and about 1 percent by weight, inclusive (i.e., between about 0.3 mg/g and 10 mg/g), of the total weight of the composition.

In certain embodiments, the solubility of the TZD or pharmaceutically acceptable salt thereof in the vehicle is at least 1 part TZD or pharmaceutically acceptable salt thereof per 3000 parts of vehicle, at least 1 part TZD or pharmaceutically acceptable salt thereof per 1000 parts of vehicle, at least 1 part TZD or pharmaceutically acceptable salt thereof per 500 parts of vehicle, at least 1 part TZD or pharmaceutically acceptable salt thereof per 300 parts of vehicle, or at least 1 part TZD or pharmaceutically acceptable salt thereof per 100 parts of vehicle. In certain embodiments, the solubility of the TZD or pharmaceutically acceptable salt thereof (expressed as part TZD:part vehicle) is between about 1:19 and about 1:3333, between about 1:19 and about 1:100, between about 1:19 and about 1:300, between about 1:100 and about 1:300, between about 1:100 and about 1000, between about 1:300 and about 1:3333, or between about 1:1000 and 1:3333, inclusive.

In certain embodiments, the alcohol may comprise a mixture of different alcohols, or just one type of alcohol. In certain embodiments, the alcohol is a monohydroxy alcohol. In certain embodiments, the monohydroxy alcohol is selected from the group consisting of methanol, ethanol, isopropanol, and n-propanol. In certain embodiments, the monohydroxy alcohol is present at a concentration between about 30 percent and about 99 percent by weight, inclusive, e.g., between about 50 percent and about 80 percent by weight, inclusive.

In certain embodiments, the oleic acid is present at a concentration between about 1 and about 5 percent by weight, inclusive.

In certain embodiments, the composition further comprises a dihydroxy alcohol, e.g., propylene glycol. In certain embodiments, the dihydroxy alcohol is present at a concentration between about 5 and about 50 percent by weight, inclusive, e.g., between about 20 and about 30 percent by weight, inclusive.

In certain embodiments, the composition further comprises glycerin. In certain embodiments, the glycerin is present at a concentration between about 0.1 and about 30 percent by weight, inclusive.

In certain embodiments, the composition further comprises water. In certain embodiments, the water is present at a concentration between about 0.1 and about 30 percent by weight, inclusive.

In certain embodiments, the composition is anhydrous (e.g., comprises between 0% to about 1% water, inclusive).

In certain embodiment, the composition is a gel.

In another aspect, provided is a process for preparing a composition as described herein, comprising the steps of: (1) combining oleic acid and an alcohol (e.g., a monohydroxy alcohol) to form a first mixture; and (2) combining a TZD or pharmaceutically acceptable salt thereof in the first mixture to provide a second mixture. In certain embodiments, any of the mixtures further comprises a viscosity-enhancing agent or buffering agent.

In another aspect, provided are methods for treating a medical condition or ameliorating a cosmetic condition in a body of a subject in need thereof, the method comprising applying to the skin of the body one or more of the compositions described herein. The medical or cosmetic condition can be, e.g., a deficiency of adipose tissue, e.g., a deficiency of subcutaneous fat.

The details of one or more embodiments of the invention are set forth in the accompanying Figures below. Other features, objects, and advantages of the invention will be apparent from the Detailed Description, Examples, and the Claims.

### Definitions

The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

Compounds described herein can comprise one or more asymmetric centers, and thus can exist in various stereoisomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, E. L. Stereochemistry of Carbon Compounds (McGraw-Hill, NY, 1962); and Wilen, S. H. Tables of Resolving Agents and Optical Resolutions p. 268 (E. L. Eliel, Ed., Univ. of Notre Dame Press, Notre Dame, Ind. 1972). The invention additionally encompasses compounds as individual isomers substantially free of other isomers, and alternatively, as mixtures of various isomers.

When a range of values is listed, it is intended to encompass each value and sub-range within the range. For example “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

As used herein, “alkyl” refers to a radical of a straight-chain or branched saturated hydrocarbon group having from 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl(C₁), ethyl(C₂), n-propyl(C₃), isopropyl(C₃), n-butyl(C₄), tert-butyl(C₄), sec-butyl(C₄), iso-butyl(C₄), n-pentyl(C₅), 3-pentanyl(C₅), amyl(C₅), neopentyl(C₅), 3-methyl-2-butanyl(C₅), tertiary amyl(C₅), and n-hexyl(C₆). Additional examples of alkyl groups include n-heptyl(C₇), n-octyl(C₈) and the like. Unless otherwise specified, each instance of an alkyl group is independently unsubstituted (an “unsubstituted alkyl”) or substituted (a “substituted alkyl”) with one or more substituents. In certain embodiments, the alkyl group is an unsubstituted C₁₋₁₀ alkyl (e.g., —CH₃). In certain embodiments, the alkyl group is a substituted C₁₋₁₀ alkyl.

As used herein, “haloalkyl” is a substituted alkyl group as defined herein wherein one or more of the hydrogen atoms are independently replaced by a halogen, e.g., fluoro, bromo, chloro, or iodo. “Perhaloalkyl” is a subset of haloalkyl, and refers to an alkyl group wherein all of the hydrogen atoms are independently replaced by a halogen, e.g., fluoro, bromo, chloro, or iodo. In some embodiments, the haloalkyl moiety has 1 to 8 carbon atoms (“C₁₋₈ haloalkyl”). In some embodiments, the haloalkyl moiety has 1 to 6 carbon atoms (“C₁₋₆ haloalkyl”). In some embodiments, the haloalkyl moiety has 1 to 4 carbon atoms (“C₁₋₄ haloalkyl”). In some embodiments, the haloalkyl moiety has 1 to 3 carbon atoms (“C₁₋₃ haloalkyl”). In some embodiments, the haloalkyl moiety has 1 to 2 carbon atoms (“C₁₋₂ haloalkyl”). In some embodiments, all of the haloalkyl hydrogen atoms are replaced with fluoro to provide a perfluoroalkyl group. In some embodiments, all of the haloalkyl hydrogen atoms are replaced with chloro to provide a “perchloroalkyl” group. Examples of haloalkyl groups include —CF₃, —CF₂CF₃, —CF₂CF₂CF₃, —CCl₃, —CFCl₂, —CF₂Cl, and the like.

As used herein, “heteroalkyl” refers to an alkyl group as defined herein which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkyl group refers to a saturated group having from 1 to 10 carbon atoms and 1 or more heteroatoms within the parent chain (“heteroC₁₋₁₀ alkyl”). In some embodiments, a heteroalkyl group is a saturated group having 1 to 9 carbon atoms and 1 or more heteroatoms within the parent chain (“heteroC₁₋₉ alkyl”). In some embodiments, a heteroalkyl group is a saturated group having 1 to 8 carbon atoms and 1 or more heteroatoms within the parent chain (“heteroC₁₋₈ alkyl”). In some embodiments, a heteroalkyl group is a saturated group having 1 to 7 carbon atoms and 1 or more heteroatoms within the parent chain (“heteroC₁₋₇ alkyl”). In some embodiments, a heteroalkyl group is a saturated group having 1 to 6 carbon atoms and 1 or more heteroatoms within the parent chain (“heteroC₁₋₆ alkyl”). In some embodiments, a heteroalkyl group is a saturated group having 1 to 5 carbon atoms and 1 or 2 heteroatoms within the parent chain (“heteroC₁₋₅ alkyl”). In some embodiments, a heteroalkyl group is a saturated group having 1 to 4 carbon atoms and for 2 heteroatoms within the parent chain (“heteroC₁₋₄ alkyl”). In some embodiments, a heteroalkyl group is a saturated group having 1 to 3 carbon atoms and 1 heteroatom within the parent chain (“heteroC₁₋₃ alkyl”). In some embodiments, a heteroalkyl group is a saturated group having 1 to 2 carbon atoms and 1 heteroatom within the parent chain (“heteroC₁₋₂ alkyl”). In some embodiments, a heteroalkyl group is a saturated group having 1 carbon atom and 1 heteroatom (“heteroC₁ alkyl”). In some embodiments, a heteroalkyl group is a saturated group having 2 to 6 carbon atoms and 1 or 2 heteroatoms within the parent chain (“heteroC₂₋₆ alkyl”). Unless otherwise specified, each instance of a heteroalkyl group is independently unsubstituted (an “unsubstituted heteroalkyl”) or substituted (a “substituted heteroalkyl”) with one or more substituents. In certain embodiments, the heteroalkyl group is an unsubstituted heteroC₁₋₁₀ alkyl. In certain embodiments, the heteroalkyl group is a substituted heteroC₁₋₁₀ alkyl.

As used herein, “alkenyl” refers to a radical of a straight-chain or branched hydrocarbon group having from 2 to 10 carbon atoms and one or more carbon-carbon double bonds (e.g., 1, 2, 3, or 4 double bonds). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl(C₂), 1-propenyl(C₃), 2-propenyl(C₃), 1-butenyl(C₄), 2-butenyl(C₄), butadienyl(C₄), and the like. Examples of C₂₋₆ alkenyl groups include the aforementioned C₂₋₄ alkenyl groups as well as pentenyl(C₅), pentadienyl(C₅), hexenyl(C₆), and the like. Additional examples of alkenyl include heptenyl(C₇), octenyl(C₈), octatrienyl(C₈), and the like. Unless otherwise specified, each instance of an alkenyl group is independently unsubstituted (an “unsubstituted alkenyl”) or substituted (a “substituted alkenyl”) with one or more substituents. In certain embodiments, the alkenyl group is an unsubstituted C₂₋₁₀ alkenyl. In certain embodiments, the alkenyl group is a substituted C₂₋₁₀ alkenyl.

As used herein, “heteroalkenyl” refers to an alkenyl group as defined herein which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkenyl group refers to a group having from 2 to 10 carbon atoms, at least one double bond, and 1 or more heteroatoms within the parent chain (“heteroC₂₋₁₀ alkenyl”). In some embodiments, a heteroalkenyl group has 2 to 9 carbon atoms at least one double bond, and 1 or more heteroatoms within the parent chain (“heteroC₂₋₉ alkenyl”). In some embodiments, a heteroalkenyl group has 2 to 8 carbon atoms, at least one double bond, and 1 or more heteroatoms within the parent chain (“heteroC₂₋₈ alkenyl”). In some embodiments, a heteroalkenyl group has 2 to 7 carbon atoms, at least one double bond, and 1 or more heteroatoms within the parent chain (“heteroC₂₋₇ alkenyl”). In some embodiments, a heteroalkenyl group has 2 to 6 carbon atoms, at least one double bond, and 1 or more heteroatoms within the parent chain (“heteroC₂₋₆ alkenyl”). In some embodiments, a heteroalkenyl group has 2 to 5 carbon atoms, at least one double bond, and 1 or 2 heteroatoms within the parent chain (“heteroC₂₋₅ alkenyl”). In some embodiments, a heteroalkenyl group has 2 to 4 carbon atoms, at least one double bond, and 1 or 2 heteroatoms within the parent chain (“heteroC₂₋₄ alkenyl”). In some embodiments, a heteroalkenyl group has 2 to 3 carbon atoms, at least one double bond, and 1 heteroatom within the parent chain (“heteroC₂₋₃ alkenyl”). In some embodiments, a heteroalkenyl group has 2 to 6 carbon atoms, at least one double bond, and 1 or 2 heteroatoms within the parent chain (“heteroC₂₋₆ alkenyl”). Unless otherwise specified, each instance of a heteroalkenyl group is independently unsubstituted (an “unsubstituted heteroalkenyl”) or substituted (a “substituted heteroalkenyl”) with one or more substituents. In certain embodiments, the heteroalkenyl group is an unsubstituted heteroC₂₋₁₀ alkenyl. In certain embodiments, the heteroalkenyl group is a substituted heteroC₂₋₁₀ alkenyl.

As used herein, “alkynyl” refers to a radical of a straight-chain or branched hydrocarbon group having from 2 to 10 carbon atoms and one or more carbon-carbon triple bonds (e.g., 1, 2, 3, or 4 triple bonds) (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl(C₂), 1-propynyl(C₃), 2-propynyl(C₃), 1-butynyl(C₄), 2-butynyl(C₄), and the like. Examples of C₂₋₆ alkenyl groups include the aforementioned C₂₋₄ alkynyl groups as well as pentynyl(C₅), hexynyl(C₆), and the like. Additional examples of alkynyl include heptynyl(C₇), octynyl(C₈), and the like. Unless otherwise specified, each instance of an alkynyl group is independently unsubstituted (an “unsubstituted alkynyl”) or substituted (a “substituted alkynyl”) with one or more substituents. In certain embodiments, the alkynyl group is an unsubstituted C₂₋₁₀ alkynyl. In certain embodiments, the alkynyl group is a substituted C₂₋₁₀ alkynyl.

As used herein, “heteroalkynyl” refers to an alkynyl group as defined herein which further includes at least one heteroatom (e.g., 1, 2, 3, or 4 heteroatoms) selected from oxygen, nitrogen, or sulfur within (i.e., inserted between adjacent carbon atoms of) and/or placed at one or more terminal position(s) of the parent chain. In certain embodiments, a heteroalkynyl group refers to a group having from 2 to 10 carbon atoms, at least one triple bond, and 1 or more heteroatoms within the parent chain (“heteroC₂₋₁₀ alkynyl”). In some embodiments, a heteroalkynyl group has 2 to 9 carbon atoms, at least one triple bond, and 1 or more heteroatoms within the parent chain (“heteroC₂₋₉ alkynyl”). In some embodiments, a heteroalkynyl group has 2 to 8 carbon atoms, at least one triple bond, and 1 or more heteroatoms within the parent chain (“heteroC₂₋₈ alkynyl”). In some embodiments, a heteroalkynyl group has 2 to 7 carbon atoms, at least one triple bond, and 1 or more heteroatoms within the parent chain (“heteroC₂₋₇ alkynyl”). In some embodiments, a heteroalkynyl group has 2 to 6 carbon atoms, at least one triple bond, and 1 or more heteroatoms within the parent chain (“heteroC₂₋₆ alkynyl”). In some embodiments, a heteroalkynyl group has 2 to 5 carbon atoms, at least one triple bond, and 1 or 2 heteroatoms within the parent chain (“heteroC₂₋₅ alkynyl”). In some embodiments, a heteroalkynyl group has 2 to 4 carbon atoms, at least one triple bond, and for 2 heteroatoms within the parent chain (“heteroC₂₋₄ alkynyl”). In some embodiments, a heteroalkynyl group has 2 to 3 carbon atoms, at least one triple bond, and 1 heteroatom within the parent chain (“heteroC₂₋₃ alkynyl”). In some embodiments, a heteroalkynyl group has 2 to 6 carbon atoms, at least one triple bond, and 1 or 2 heteroatoms within the parent chain (“heteroC₂₋₆ alkynyl”). Unless otherwise specified, each instance of a heteroalkynyl group is independently unsubstituted (an “unsubstituted heteroalkynyl”) or substituted (a “substituted heteroalkynyl”) with one or more substituents. In certain embodiments, the heteroalkynyl group is an unsubstituted heteroC₂₋₁₀ alkynyl. In certain embodiments, the heteroalkynyl group is a substituted heteroC₂₋₁₀ alkynyl.

As used herein, “carbocyclyl” or “carbocyclic” refers to a radical of a non-aromatic cyclic hydrocarbon group having from 3 to 10 ring carbon atoms (“C₃₋₁₀ carbocyclyl”) and zero heteroatoms in the nonaromatic ring system. In some embodiments, a carbocyclyl group has 3 to 8 ring carbon atoms (“C₃₋₈ carbocyclyl”). In some embodiments, a carbocyclyl group has 3 to 7 ring carbon atoms (“C₃₋₇ carbocyclyl”). In some embodiments, a carbocyclyl group has 3 to 6 ring carbon atoms (“C₃₋₆ carbocyclyl”). In some embodiments, a carbocyclyl group has 4 to 6 ring carbon atoms (“C₄₋₆ carbocyclyl”). In some embodiments, a carbocyclyl group has 5 to 6 ring carbon atoms (“C₅₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include, without limitation, cyclopropyl(C₃), cyclopropenyl(C₃), cyclobutyl(C₄), cyclobutenyl(C₄), cyclopentyl(C₅), cyclopentenyl(C₅), cyclohexyl(C₆), cyclohexenyl(C₆), cyclohexadienyl(C₆), and the like. Exemplary C₃₋₈ carbocyclyl groups include, without limitation, the aforementioned C₃₋₆ carbocyclyl groups as well as cycloheptyl(C₇), cycloheptenyl(C₇), cycloheptadienyl(C₇), cycloheptatrienyl(C₇), cyclooctyl(C₈), cyclooctenyl(C₈), bicyclo[2.2.1]heptanyl(C₇), bicyclo[2.2.2]octanyl(C₈), and the like. Exemplary C₃₋₁₀ carbocyclyl groups include, without limitation, the aforementioned C₃₋₈ carbocyclyl groups as well as cyclononyl(C₉), cyclononenyl(C₉), cyclodecyl(C₁₀), cyclodecenyl(C₁₀), octahydro-1H-indenyl(C₉), decahydronaphthalenyl(C₁₀), spiro[4.5]decanyl(C₁₀), and the like. As the foregoing examples illustrate, in certain embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and can be saturated or can contain one or more carbon-carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently unsubstituted (an “unsubstituted carbocyclyl”) or substituted (a “substituted carbocyclyl”) with one or more substituents. In certain embodiments, the carbocyclyl group is an unsubstituted C₃₋₁₀ carbocyclyl. In certain embodiments, the carbocyclyl group is a substituted C₃₋₁₀ carbocyclyl.

In some embodiments, “carbocyclyl” is a monocyclic, saturated carbocyclyl group having from 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 4 to 6 ring carbon atoms (“C₄₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl(C₅) and cyclohexyl(C₅). Examples of C₃₋₆ cycloalkyl groups include the aforementioned C₅₋₆ cycloalkyl groups as well as cyclopropyl(C₃) and cyclobutyl(C₄). Examples of C₃₋₈ cycloalkyl groups include the aforementioned C₃₋₆ cycloalkyl groups as well as cycloheptyl(C₇) and cyclooctyl(C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is an unsubstituted C₃₋₁₀ cycloalkyl. In certain embodiments, the cycloalkyl group is a substituted C₃₋₁₀ cycloalkyl.

As used herein, “heterocyclyl” or “heterocyclic” refers to a radical of a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and can be saturated or can contain one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently unsubstituted (an “unsubstituted heterocyclyl”) or substituted (a “substituted heterocyclyl”) with one or more substituents. In certain embodiments, the heterocyclyl group is an unsubstituted 3-14 -membered heterocyclyl. In certain embodiments, the heterocyclyl group is a substituted 3-14 -membered heterocyclyl.

In some embodiments, a heterocyclyl group is a 5-10 -membered non-aromatic ring system having ring carbon atoms and 1-4 ring heteroatoms, wherein each heteroatom is independently selected from nitrogen, oxygen, and sulfur (“5-10 -membered heterocyclyl”). In some embodiments, a heterocyclyl group is a 5-8 -membered nonaromatic ring system having ring carbon atoms and 1-4 ring heteroatoms, wherein each heteroatom is independently selected from nitrogen, oxygen, and sulfur (“5-8 -membered heterocyclyl”). In some embodiments, a heterocyclyl group is a 5-6 -membered nonaromatic ring system having ring carbon atoms and 1-4 ring heteroatoms, wherein each heteroatom is independently selected from nitrogen, oxygen, and sulfur (“5-6 -membered heterocyclyl”). In some embodiments, the 5-6 -membered heterocyclyl has 1-3 ring heteroatoms selected from nitrogen, oxygen, and sulfur. In some embodiments, the 5-6 -membered heterocyclyl has 1-2 ring heteroatoms selected from nitrogen, oxygen, and sulfur. In some embodiments, the 5-6 -membered heterocyclyl has 1 ring heteroatom selected from nitrogen, oxygen, and sulfur.

Exemplary 3-membered heterocyclyl groups containing 1 heteroatom include, without limitation, azirdinyl, oxiranyl, thiiranyl. Exemplary 4-membered heterocyclyl groups containing 1 heteroatom include, without limitation, azetidinyl, oxetanyl and thietanyl. Exemplary 5-membered heterocyclyl groups containing 1 heteroatom include, without limitation, tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyl groups containing 2 heteroatoms include, without limitation, dioxolanyl, oxathiolanyl and dithiolanyl. Exemplary 5-membered heterocyclyl groups containing 3 heteroatoms include, without limitation, triazolinyl, oxadiazolinyl, and thiadiazolinyl. Exemplary 6-membered heterocyclyl groups containing 1 heteroatom include, without limitation, piperidinyl, tetrahydropyranyl, dihydropyranyl, dihydropyridinyl, and thianyl. Exemplary 6-membered heterocyclyl groups containing 2 heteroatoms include, without limitation, piperazinyl, morpholinyl, dithianyl, dioxanyl. Exemplary 6-membered heterocyclyl groups containing 3 heteroatoms include, without limitation, triazinanyl. Exemplary 7-membered heterocyclyl groups containing 1 heteroatom include, without limitation, azepanyl, oxepanyl and thiepanyl. Exemplary 8-membered heterocyclyl groups containing 1 heteroatom include, without limitation, azocanyl, oxecanyl and thiocanyl. Exemplary bicyclic heterocyclyl groups include, without limitation, indolinyl, isoindolinyl, dihydrobenzofuranyl, dihydrobenzothienyl, tetrahydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydropyrano[3,4-b]pyrrolyl, 5,6-dihydro-4H-furo[3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo[2,3-b]pyridinyl, 4,5,6,7-tetrahydrofuro[3,2-c]pyridinyl, 4,5,6,7-tetrahydrothieno[3,2-b]pyridinyl, 1,2,3,4-tetrahydro-1,6-naphthyridinyl, and the like.

As used herein, “aryl” refers to a radical of a monocyclic or polycyclic (e.g., bicyclic or tricyclic) 4n+2 aromatic ring system (e.g., having 6, 10, or 14π electrons shared in a cyclic array) having 6-14 ring carbon atoms and zero heteroatoms provided in the aromatic ring system (“C₆₋₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl and 2-naphthyl). In some embodiments, an aryl group has 14 ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently unsubstituted (an “unsubstituted aryl”) or substituted (a “substituted aryl”) with one or more substituents. In certain embodiments, the aryl group is an unsubstituted C₆₋₁₄ aryl. In certain embodiments, the aryl group is a substituted C₆₋₁₄ aryl.

As used herein, “heteroaryl” refers to a radical of a 5-14 membered monocyclic or polycyclic (e.g., bicyclic, tricyclic) 4n+2 aromatic ring system (e.g., having 6, 10, or 14π electrons shared in a cyclic array) having ring carbon atoms and 1-4 ring heteroatoms provided in the aromatic ring system, wherein each heteroatom is independently selected from nitrogen, oxygen and sulfur (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently unsubstituted (an “unsubstituted heteroaryl”) or substituted (a “substituted heteroaryl”) with one or more substituents. In certain embodiments, the heteroaryl group is an unsubstituted 5-14 membered heteroaryl. In certain embodiments, the heteroaryl group is a substituted 5-14 membered heteroaryl.

Exemplary 5-membered heteroaryl groups containing 1 heteroatom include, without limitation, pyrrolyl, furanyl and thiophenyl. Exemplary 5-membered heteroaryl groups containing 2 heteroatoms include, without limitation, imidazolyl, pyrazolyl, oxazolyl, isoxazolyl, thiazolyl, and isothiazolyl. Exemplary 5-membered heteroaryl groups containing 3 heteroatoms include, without limitation, triazolyl, oxadiazolyl, and thiadiazolyl. Exemplary 5-membered heteroaryl groups containing 4 heteroatoms include, without limitation, tetrazolyl. Exemplary 6-membered heteroaryl groups containing 1 heteroatom include, without limitation, pyridinyl. Exemplary 6-membered heteroaryl groups containing 2 heteroatoms include, without limitation, pyridazinyl, pyrimidinyl, and pyrazinyl. Exemplary 6-membered heteroaryl groups containing 3 or 4 heteroatoms include, without limitation, triazinyl and tetrazinyl, respectively. Exemplary 7-membered heteroaryl groups containing 1 heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl. Exemplary tricyclic heteroaryl groups include, without limitation, phenanthridinyl, dibenzofuranyl, carbazolyl, acridinyl, phenothiazinyl, phenoxazinyl and phenazinyl.

Affixing the suffix “-ene” to a group indicates the group is a divalent moiety, e.g., alkylene is the divalent moiety of alkyl, alkenylene is the divalent moiety of alkenyl, alkynylene is the divalent moiety of alkynyl, heteroalkylene is the divalent moiety of heteroalkyl, heteroalkenylene is the divalent moiety of heteroalkenyl, heteroalkynylene is the divalent moiety of heteroalkynyl, carbocyclylene is the divalent moiety of carbocyclyl, heterocyclylene is the divalent moiety of heterocyclyl, arylene is the divalent moiety of aryl, and heteroarylene is the divalent moiety of heteroaryl.

As understood from the above, alkyl, alkenyl, alkynyl, heteroalkyl, heteroalkenyl, heteroalkynyl, carbocyclyl, heterocyclyl, aryl, and heteroaryl groups, as defined herein, are, in certain embodiments, optionally substituted. Optionally substituted refers to a group which may be substituted or unsubstituted (e.g., “substituted” or “unsubstituted” alkyl, “substituted” or “unsubstituted” alkenyl, “substituted” or “unsubstituted” alkynyl, “substituted” or “unsubstituted” heteroalkyl, “substituted” or “unsubstituted” heteroalkenyl, “substituted” or “unsubstituted” heteroalkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted” means that at least one hydrogen present on a group is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, and includes any of the substituents described herein that results in the formation of a stable compound. The present invention contemplates any and all such combinations in order to arrive at a stable compound. For purposes of this invention, heteroatoms such as nitrogen may have hydrogen substituents and/or any suitable substituent as described herein which satisfy the valencies of the heteroatoms and results in the formation of a stable moiety.

Exemplary substituents include, but are not limited to, halogen, —CN, —NO₂, —N₃, —SO₂H, —SO₃H, —OH, —OR^{aa}, —ON(R^{bb})₂, —N(R^{bb})₂, —N(R^{bb})₃⁺X⁻, —N(OR^{cc})R^{bb}, —SH, —SR^{aa}, —SSR^{cc}, —C(═O)R^{aa}, —CO₂H, —CHO, —C(OR^{cc})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})_{c}—C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)₂R^{aa}, —OP(═O)₂R^{aa}, —P(═O)(R^{aa})₂, —OP(═O)(OR^{cc})₂, —P(═O)₂N(R^{bb})₂, —OP(═O)₂N(R^{bb})₂, —P(═O)(NR^{bb})₂, —OP(═O)(NR^{bb})₂, —NR^{bb}P(═O)(OR^{cc})₂, —NR^{bb}P(═O)(NR^{bb})₂, —P(R^{cc})₂, —P(R^{cc})₃, —OP(R^{cc})₂, —OP(R^{cc})₃, —B(R^{aa})₂, —B(OR^{cc})₂, —BR^{aa}(OR^{cc}), C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;

or two geminal hydrogens on a carbon atom are replaced with the group ═O, ═S, ═NN(R^{bb})₂, ═NNR^{bb}C(═O)R^{aa}, ═NNR^{bb}C(═O)OR^{aa}, ═NNR^{bb}S(═O)₂R^{aa}, ═NR^{bb}, or ═NOR^{cc};

each instance of R^{aa} is, independently, selected from C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀alkenyl, heteroC₂₋₁₀alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;

each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)₂R^{aa}, —P(═O)(R^{aa})₂, —P(═O)₂N(R^{cc})₂, —P(═O)(NR^{cc})₂, C₁₋₁₀ alkyl, C₁₋₁₀ perhaloalkyl, C₂₋₁₀ alkenyl, C₂₋₁₀ alkynyl, heteroC₁₋₁₀alkyl, heteroC₂₋₁₀alkenyl, heteroC₂₋₁₀alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;

each instance of R^{cc} is, independently, selected from hydrogen, C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;

each instance of R^{dd} is, independently, selected from halogen, —CN, —NO₂, —N₃, —SO₂H, —SO₃H, —OH, —OR^{cc}, —ON(R^{ff})₂, —N(R^{ff})₂, —N(R^{ff})₃⁺X⁻, —N(OR^{cc})R^{ff}, —SH, —SR^{cc}, —SSR^{cc}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)₂R^{ee}, —P(═O)(R^{ee})₂, —OP(═O)(R^{ee})₂, —OP(═O)(OR^{ee})₂, C₁₋₆ alkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl, C₃₋₁₀ carbocyclyl, 3-10 membered heterocyclyl, C₆₋₁₀ aryl, 5-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups, or two geminal R^{dd} substituents can be joined to form ═O or ═S;

each instance of R^{ee} is, independently, selected from C₁₋₆ alkyl, C₁₋₆ perhaloalkyl, C₂₋₆ alkenyl, C₂₋₆ alkynyl, heteroC₁₋₆ alkyl, heteroC₂₋₆alkenyl, heteroC₂₋₆ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;

each instance of R^{ff} is, independently, selected from hydrogen, C₁₋₆ alkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl, C₃₋₁₀ carbocyclyl, 3-10 membered heterocyclyl, C₆₋₁₀ aryl and 5-10 membered heteroaryl, or two R^{ff} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups; and

each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)⁺X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C₁₋₆ alkyl)₃, —OSi(C₁₋₆ alkyl)₃-C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)₂(C₁₋₆ alkyl), —P(═O)(C₁₋₆ alkyl)₂, —OP(═O)(C₁₋₆ alkyl)₂, —OP(═O)(OC₁₋₆ alkyl)₂, C₁₋₆ alkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl, C₃₋₁₀ carbocyclyl, C₆₋₁₀ aryl, 3-10 membered heterocyclyl, 5-10 membered heteroaryl; or two geminal R^{gg} substituents can be joined to form ═O or ═S; wherein X⁻ is a counterion.

In certain embodiments, the substituent includes, but is not limited to, halogen, —CN, —NO₂, —N₃, —SO₂H, —SO₃H, —OH, —OR^{aa}, —N(R^{bb})₂, —SH, —SR^{aa}, —C(═O)R^{aa}, —CO₂H, —CHO, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═O)NR^{bb}SO₂R^{aa}, —NR^{bb}SO₂R^{aa}, —SO₂N(R^{bb})₂, —SO₂R^{aa}, —S(═O)R^{aa}, —Si(R^{aa})₃, C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups.

As used herein, the term “halo” or “halogen” refers to fluorine (fluoro, —F), chlorine (chloro, —Cl), bromine (bromo, —Br), or iodine (iodo, —I).

As used herein, a “counterion” is a negatively charged group associated with a positively charged quarternary amine in order to maintain electronic neutrality. Exemplary counterions include halide ions (e.g., F⁻, Cl⁻, Br⁻, I⁻), NO₃⁻, ClO₄⁻, OH⁻, H₂PO₄⁻, HSO₄⁻, sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), and carboxylate ions (e.g., acetate, ethanoate, propanoate, benzoate, glycerate, lactate, tartrate, glycolate, and the like).

In certain embodiments, the substituent present on the nitrogen atom is an nitrogen protecting group (also referred to herein as an “amino protecting group”). Nitrogen protecting groups include, but are not limited to, —OH, —OR^{aa}, —N(R^{cc})₂, —C(═O)R^{aa}, —C(═O)N(R^{cc})₂, —CO₂R^{aa}, —SO₂R^{aa}, —C(═NR^{cc})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —C₁₋₁₀ alkyl (e.g., aralkyl, heteroaralkyl), C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl groups, wherein each alkyl, alkenyl, alkynyl, heteroalkyl, heteroalkenyl, heteroalkynyl, carbocyclyl, heterocyclyl, aralkyl, aryl, and heteroaryl is independently substituted with 0, 1, 2, 3, 4, or 5 R^{dd} groups, and wherein R^{aa}, R^{bb}, R^{cc} and R^{dd} are as defined herein. Nitrogen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

For example, nitrogen protecting groups such as amide groups (e.g., —C(═O)R^{aa}) include, but are not limited to, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxyacylamino)acetamide, 3-(p-hydroxyphenyl)propanamide, 3-(o-nitrophenyl)propanamide, 2methyl-2-(o-nitrophenoxy)propanamide, 2methyl-2-(o-phenylazophenoxy)propanamide, 4-chlorobutanamide, 3-methyl-3-nitrobutanamide, o-nitrocinnamide, N-acetylmethionine derivative, o-nitrobenzamide and o-(benzoyloxymethyl)benzamide.

Nitrogen protecting groups such as carbamate groups (e.g., —C(═O)OR^{aa}) include, but are not limited to, methyl carbamate, ethyl carbamante, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

Nitrogen protecting groups such as sulfonamide groups (e.g., —S(═O)₂R^{aa}) include, but are not limited to, p-toluenesulfonamide (Ts), benzenesulfonamide, 2,3,6,-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

Other nitrogen protecting groups include, but are not limited to, phenothiazinyl(10)-acyl derivative, N′-p-toluenesulfonylaminoacyl derivative, N′-phenylaminothioacyl derivative, N-benzoylphenylalanyl derivative, N-acetylmethionine derivative, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N-(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentaacylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys).

As used herein, the term “prodrug” means a compound that can hydrolyze, oxidize, or otherwise react under biological conditions (e.g., in vitro or in vivo enzymatic conditions) to provide a pharmacologically active compound. In certain cases, a prodrug has improved physical and/or delivery properties over the parent compound. Prodrugs are typically designed to enhance pharmacologically, pharmaceutically and/or pharmacokinetically based properties associated with the parent compound. The advantage of a prodrug can lie in its physical properties, such as enhanced solubility for formulation compared to the parent compound, or enhanced penetration across the stratum corneum of the skin, or enhanced deposition in the subcutaneous fat.

The term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, Berge et al., describes pharmaceutically acceptable salts in detail in J. Pharmaceutical Sciences (1977) 66:1-19. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Pharmaceutically acceptable salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

Unless otherwise specified, “disease”, “disorder,” and “condition” are used interchangeably herein. Conditions for which treatment and prevention are contemplated may be further classified as a medical condition or a cosmetic condition. A “medical condition,” as used herein, refers to an abnormal condition that affects the body. A “cosmetic condition,” as used herein, refers to a condition other than a medical condition that affects the physical appearance of the body. A cosmetic condition can occur, for example, due to normal processes in a body, such as aging, pregnancy, puberty, and exposure to the sun or the elements, or due to normal features of a body, such as inherited facial features or body shapes that are found in healthy individuals. Various medical and cosmetic conditions are described herein. A “cosmetic composition” is contemplated useful for such purpose. A “therapeutic method” refers to a method or procedure intended to treat or prevent a medical condition, and a “pharmaceutical composition” is contemplated useful for such purpose.

As used herein, an “individual” or “subject” to which administration is contemplated includes, but is not limited to, humans (i.e., a male or female of any age group, e.g., a pediatric subject (e.g., child, adolescent) or adult subject (e.g., young adult, middle-aged adult or senior adult)), other primates (e.g., cynomolgus monkeys, rhesus monkeys) and commercially relevant mammals such as cattle, pigs, horses, sheep, goats, cats, and/or dogs. In any aspect and/or embodiment of the invention, the mammal is a human.

As used herein, “local administration” or “administering locally” or “local effect” means administration/application of the active ingredient (i.e., TZD or pharmaceutically acceptable salt thereof) directly, or in proximity to, a part of the body, tissue, or lesion where said active substance is intended to exert its action. This may include, for example, topical administration to a part of the skin.

As used herein, unless otherwise specified, “topical administration” or “topically” means application to the surface of the skin, e.g., in a non-invasive manner.

As used herein, and unless otherwise specified, a “therapeutically effective amount” “an amount sufficient” or “sufficient amount” of a compound (i.e., TZD or pharmaceutically acceptable salt thereof) means the level, amount or concentration of the compound needed to treat a disease, disorder or condition. The term “therapeutically effective amount” can encompass an amount that improves overall therapy, reduces or avoids symptoms or causes of disease or condition, or enhances the therapeutic efficacy of another therapeutically active agent.

### BRIEF DESCRIPTION OF THE DRAWINGS

FIG. 1A-1C depicts a schematic diagram of superficial skin delivery (FIG. 1A), transdermal delivery into the bloodstream (FIG. 1B), and percutaneous delivery into fat (FIG. 1C). The relative presence or concentration of a drug is indicated by the letter “D.” Note that with superficial skin delivery (FIG. 1A), the drug remains on or within the skin. With transdermal delivery (FIG. 1B), substantial amounts of the drug are absorbed in the dermal circulation and into the bloodstream, thus leading to systemic effects. With percutaneous delivery (FIG. 1C), drug passes into the subcutaneous fat, with negligible presence in the bloodstream.

### DETAILED DESCRIPTION OF CERTAIN EMBODIMENTS OF THE INVENTION

The present invention arose from the discovery that addition of oleic acid to an alcohol-based vehicle (e.g., an monohydroxy alcohol-based vehicle, such as ethanol or isopropanol) substantially and unexpectedly increased the solubility of a TZD, or pharmaceutically acceptable salt thereof, in the vehicle. As described in Example 1, the addition of a relatively small amount (e.g., about 3% w/w) of oleic acid to the formulation of an alcohol-based vehicle increased the solubility of rosiglitazone maleate in the vehicle, e.g., by over 75-fold in ethanol, and about 3-fold in isopropanol. See, e.g., Table 1A of Example 1. The improved solubility made it possible to prepare suitable oleic acid/alcohol-based compositions wherein a TZD (e.g., rosiglitazone maleate or pioglitazone hydrochloride) was dissolved at concentrations of about 1 mg/g to about 10 mg/g (i.e., about 0.1% to about 10% w/w of the total weight of the composition). See, e.g., Examples 1 and 2. The compositions prepared were physically and chemically stable and effectively delivered the TZD across skin. See, e.g., Examples 1 to 3. Furthermore, when administered to the skin of an individual, the compositions were safe, well-tolerated, and effective, e.g., to locally increase subcutaneous fat. See, e.g., Examples 4.

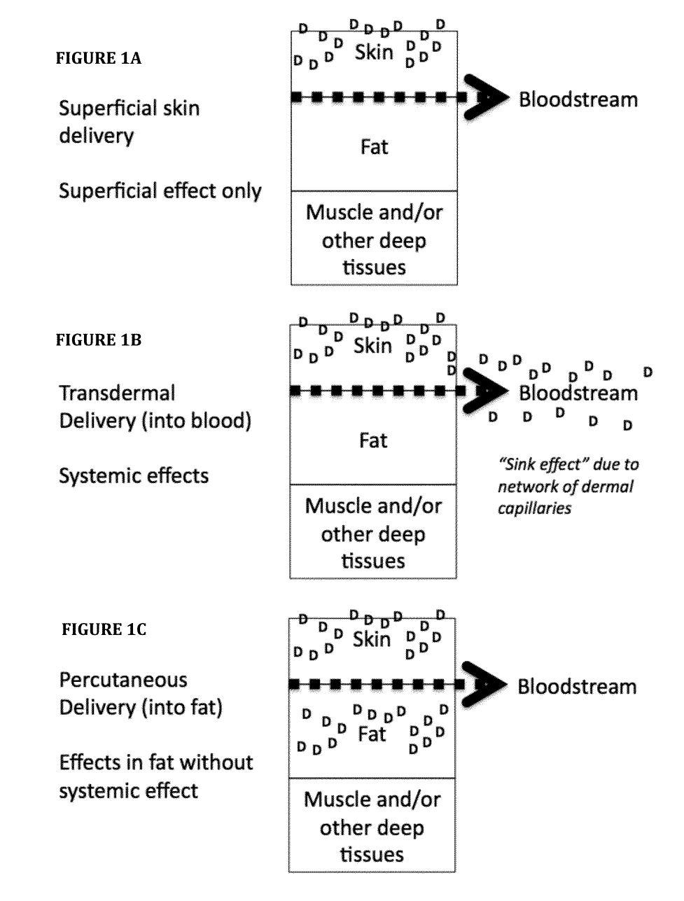

One technical problem solved by the compositions described herein was to enable percutaneous delivery of the TZD, i.e., such that the TZD applied to the skin was delivered to subcutaneous fat without a systemic effect. This percutaneous mode of delivery is to be distinguished from superficial application to the skin, whereby deep penetration does not occur. See, e.g., FIG. 1. Percutaneous administration is also to be distinguished from transdermal administration, where the objective is absorption into the bloodstream to achieve a systemic effect. Transdermal administration of a thiazolidinedione, e.g., rosiglitazone, to the skin can result in clinically significant drug delivery to the bloodstream. See, e.g., Damodharan et al, Skin permeation of rosiglitazone from transdermal matrix patches, Pharmaceutical Technology (2010) 34:56-72. See also, e.g., Ghosh et al, Feasibility of rosiglitazone maleate for transdermal delivery, Int. J. Pharm. Res. Innov. (2011) 2:23-31. Transdermal administration of thiazolidinediones is considered undesirable and potentially unsafe and would undermine the purpose of the invention, e.g., by causing obesity. While superficial and transdermal administration are common pharmaceutical routes of administration, very few examples of percutaneous administration to subcutaneous fat are known for any compound. See, e.g., Singh et al., Local deep tissue penetration of compounds after dermal application: structure-tissue penetration relationships. JPET (1996) 279:908-917.

### Thiazolidinedione Compounds

TZDs are a class of medications previously approved for systemic administration for the treatment of type 2 diabetes mellitus. TZDs specifically contemplated for use include, but are not limited to, rosiglitazone, pioglitazone, troglitazone, ciglitazone, netoglitazone, rivoglitazone, and pharmaceutically acceptable salts thereof, e.g., rosiglitazone maleate and pioglitazone hydrochloride.

In certain embodiments, the thiazolidinedione is a compound of Formula (I):

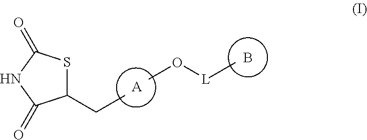

or a pharmaceutically acceptable salt or prodrug thereof;
wherein:

Ring A is substituted or unsubstituted arylene or substituted or unsubstituted heteroarylene;

L is substituted or unsubstituted C₁₋₆alkylene or substituted or unsubstituted heteroC₁₋₆alkylene; and

Ring B is substituted or unsubstituted carbocyclyl, substituted or unsubstituted heterocyclyl, substituted or unsubstituted aryl, or substituted or unsubstituted heteroaryl.

In certain embodiments, Ring A is substituted or unsubstituted arylene, e.g., substituted or unsubstituted phenylene or substituted or unsubstituted naphthylene. In certain embodiments, Ring A is substituted or unsubstituted phenylene.

In certain embodiments, Ring A is substituted or unsubstituted heteroarylene, e.g., a 6-membered heteroarylene such as substituted or unsubstituted pyridinylene.

In certain embodiments, L is substituted or unsubstituted C₁₋₆alkylene, e.g., substituted or unsubstituted C₁alkylene, substituted or unsubstituted C₂alkylene, substituted or unsubstituted C₃alkylene, substituted or unsubstituted C₄alkylene, substituted or unsubstituted C₅alkylene, or substituted or unsubstituted C₆alkylene. In certain embodiments, L is —CH₂—, —(CH₂)₂—, —(CH₂)₃—, —(CH₂)₄—, —(CH₂)₅—, or —(CH₂)₆—.

In certain embodiments, L is substituted or unsubstituted heteroC₁₋₆alkylene, e.g., substituted or unsubstituted heteroC₁alkylene, substituted or unsubstituted heteroC₂alkylene, substituted or unsubstituted heteroC₃alkylene, substituted or unsubstituted heteroC₄alkylene, substituted or unsubstituted heteroC₅alkylene, or substituted or unsubstituted heteroC₆alkylene. In certain embodiments, L is —CH₂(Y)—, —(CH₂)₂(Y)—, —(CH₂)₃(Y)—, —(CH₂)₄(Y)—, —(CH₂)₅(Y)—, or —(CH₂)₆(Y)—, wherein Y is O, S, or NR^{L}, and R^{L} is hydrogen, a nitrogen protecting group, or C₁₋₆alkyl (e.g., methyl). In certain embodiments, Y is NR^{L}, and R^{L} is hydrogen or methyl.

In certain embodiments, Ring B is substituted or unsubstituted carbocyclyl, e.g., substituted or unsubstituted C₅₋₆ carbocyclyl. In certain embodiments, Ring B is substituted or unsubstituted cyclopentyl. In certain embodiments, Ring B is substituted or unsubstituted cyclohexyl.

In certain embodiments, Ring B is substituted or unsubstituted heterocyclyl, e.g., a 5- to 6-membered heterocyclyl. In certain embodiments, the heterocyclyl ring comprises a substituted or unsubstituted aryl or substituted or unsubstituted heteroaryl ring fused thereto, wherein the point of attachment is on the heterocyclyl ring. In certain embodiments, Ring B is a 6-membered substituted or unsubstituted heterocyclyl, e.g., a substituted or unsubstituted dihydropyranyl comprising a substituted or unsubstituted aryl ring fused thereto, also referred to as substituted or unsubstituted chromanyl.

In certain embodiments, Ring B is substituted or unsubstituted aryl, e.g., substituted or unsubstituted phenyl or substituted or unsubstituted naphthyl. In certain embodiments, Ring B is substituted or unsubstituted phenyl.

In certain embodiments, Ring B is substituted or unsubstituted heteroaryl. In certain embodiments, Ring B is a substituted or unsubstituted 6-membered heteroaryl, e.g., substituted or unsubstituted pyridinyl. In certain embodiments, Ring B is a substituted or unsubstituted bicyclic heteroaryl, e.g., a substituted or unsubstituted 5,6-bicyclic heteroaryl, e.g., substituted or unsubstituted benzimidazolyl.

Various combinations of the above embodiments are contemplated herein.

For example, in certain embodiments, wherein Ring A is phenylene, provided is a compound of Formula (I-a):

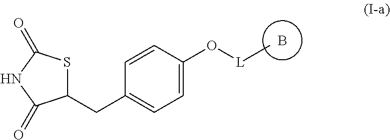

or a pharmaceutically acceptable salt or prodrug thereof. In certain embodiments, L is —CH₂(Y)— or —(CH₂)₂(Y)—, wherein Y is O, S, or NR^{L}. In certain embodiments, L is —CH₂—, —(CH₂)₂—, or —(CH₂)₃—. In certain embodiments, Ring B is substituted or unsubstituted cyclohexyl. In certain embodiments, Ring B is a substituted or unsubstituted chromanyl. In certain embodiments, Ring B is substituted or unsubstituted phenyl. In certain embodiments, Ring B is substituted or unsubstituted heteroaryl, e.g., substituted or unsubstituted pyridinyl or substituted or unsubstituted benzimidazolyl.

In certain embodiments, wherein Ring A is naphthylene, provided is a compound of Formula (I-b):

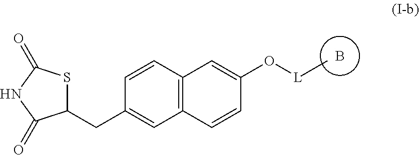

or a pharmaceutically acceptable salt or prodrug thereof In certain embodiments, L is —CH₂(Y)— or —(CH₂)₂(Y)—, wherein Y is O, S, or NR^{L}. In certain embodiments, L is —CH₂—, —(CH₂)₂—, or —(CH₂)₃—. In certain embodiments, Ring B is substituted or unsubstituted cyclohexyl. In certain embodiments, Ring B is a substituted or unsubstituted chromanyl. In certain embodiments, Ring B is substituted or unsubstituted phenyl. In certain embodiments, Ring B is substituted or unsubstituted heteroaryl, e.g., substituted or unsubstituted pyridinyl or substituted or unsubstituted benzimidazolyl.

### Pharmaceutical and Cosmetic Compositions

As generally described herein, provided are compositions for topical administration comprising an active ingredient and a pharmaceutically acceptable vehicle. As used herein, an “active ingredient” refers to a TZD, or a pharmaceutically acceptable salt or prodrug thereof, as described herein.

Furthermore, as used herein, a “composition” refers to a pharmaceutical composition (e.g., useful for treatment of a particular disease, disorder, or condition, such as diagnosed by a medical professional) or cosmetic composition (e.g., for beautification purposes). Composition and medicament may be used interchangeably herein. While pharmaceutical compositions are contemplated useful for therapeutic and prophylactic purposes, and cosmetic compositions are contemplated useful for beautification purposes, there is necessarily overlap between the two compositions in terms of use. For example, a pharmaceutical composition is also contemplated useful for beautification purposes.

In certain embodiments, the active ingredient is provided in a therapeutically effective amount in the composition.

In one aspect, provided are compositions for topical administration comprising the active ingredient, oleic acid, and one or more alcohols (e.g., monohydroxy alcohols). As used herein an alcohol is an organic alcohol of formula R¹—OH, wherein R¹ is substituted or unsubstituted C₁₋₆alkyl, substituted or unsubstituted C₂₋₆alkenyl,or substituted or unsubstituted C₂₋₆alkynyl, and encompasses monohydroxy alcohols (containing 1 hydroxyl substituent), dihydroxy alcohols (containing 2 or more hydroxyl substituents), and polyhydroxyalcohols (containing 3 or more hydroxyl substituents). In certain embodiments, a “monohydroxy alcohol” is an organic alcohol of formula R¹—OH, wherein R¹ is substituted or unsubstituted C₁₋₆alkyl, substituted or unsubstituted C₂₋₆alkenyl,or substituted or unsubstituted C₂₋₆alkynyl, wherein the alkyl, alkenyl, or alkynyl groups do not further comprise additional substitution with hydroxyl (—OH). In certain embodiments, R¹ is substituted or unsubstituted C₁₋₆alkyl, substituted or unsubstituted C₁₋₅alkyl, substituted or unsubstituted C₁₋₄alkyl, substituted or unsubstituted C₁₋₃alkyl, or substituted or unsubstituted C₁₋₂alkyl. In certain embodiments, the one or more monohydroxy alcohols is selected from the group consisting of methanol, ethanol, n-propanol, and isopropanol.

In certain embodiments, the composition further comprises one or more dihydroxy alcohols. In certain embodiments, a “dihydroxy alcohol” is an organic alcohol of formula HO—R²—OH, wherein R² is substituted or unsubstituted C₁₋₆alkylene, substituted or unsubstituted C₂₋₆alkenylene, or substituted or unsubstituted C₂₋₆alkynylene, wherein the alkylene, alkenylene, or alkynylene groups do not further comprise additional substitution with hydroxyl (—OH). In certain embodiments, R² is substituted or unsubstituted C₁₋₆alkylene, substituted or unsubstituted C₁₋₅alkylene, substituted or unsubstituted C₁₋₄alkylene, substituted or unsubstituted C₁₋₃alkylene, or substituted or unsubstituted C₁₋₂alkylene. In certain embodiments, the one or more dihydroxy alcohols is selected from the group consisting of propylene glycol, 1,3-butanediol, and ethylene glycol.

In certain embodiments, the composition further comprises glycerin. In certain embodiments, the composition further comprises water. In certain embodiments, the composition is anhydrous. In certain embodiments, the composition is a gel. In certain embodiments, the composition further comprises a viscosity-enhancing agent. In certain embodiments, the composition further comprises an antioxidant. In certain embodiments, the composition further comprises a buffering agent. In certain embodiments, the composition is not irritating to the skin.

In certain embodiments, the TZD is rosiglitazone or a pharmaceutically acceptable salt thereof. In certain embodiments, the TZD is rosiglitazone maleate. In certain embodiments, the TZD is pioglitazone or a pharmaceutically acceptable salt thereof. In certain embodiments, the TZD is pioglitazone hydrochloride. In certain embodiments, the TZD is troglitazone or a pharmaceutically acceptable salt thereof. In certain embodiments, the TZD is ciglitazone or a pharmaceutically acceptable salt thereof. In certain embodiments, the TZD is netoglitazone or a pharmaceutically acceptable salt thereof.

In some embodiments, the final concentration of the TZD or pharmaceutically acceptable salt thereof provided in the composition is between about 0.03 percent and about 5 percent (by weight), inclusive (i.e., between about 0.3 mg/g and 50 mg/g). In some embodiments, the final concentration is between about 0.03 percent and about 4 percent, between about 0.03 percent and about 3 percent, between about 0.03 percent and about 2 percent, between about 0.03 percent and about 1 percent, 0.03 and about 0.1 percent, about 0.03 and about 0.3 percent, about 0.1 and about 0.3 percent, about 0.1 and about 1 percent, about 0.3 and about 1 percent, about 0.3 and about 3 percent, about 1 and about 5 percent, about 1 and about 3 percent, about 2 and about 4 percent, or about 3 and about 5 percent (by weight), inclusive. These percentages are expressed by weight of the total weight of the composition.

In some embodiments, the final concentration of the TZD or pharmaceutically acceptable salt thereof provided in the composition is between about 0.03 percent and about 5 percent (by volume), inclusive (i.e., between about 0.3 mg/mL and 50 mg/mL. In some embodiments, the final concentration is between about 0.03 percent and about 4 percent, between about 0.03 percent and about 3 percent, between about 0.03 percent and about 2 percent, between about 0.03 percent and about 1 percent, 0.03 and about 0.1 percent, about 0.03 and about 0.3 percent, about 0.1 and about 0.3 percent, about 0.1 and about 1 percent, about 0.3 and about 1 percent, about 0.3 and about 3 percent, about 1 and about 5 percent, about 1 and about 3 percent, about 2 and about 4 percent, or about 3 and about 5 percent (weight per volume), inclusive. In this paragraph, percentages are expressed as weight of per total volume of the composition.

In some embodiments, the ratio of the TZD or pharmaceutically acceptable salt thereof to the vehicle (TZD:vehicle) is between about 1:19 and about 1:3333, between about 1:19 and about 1:100, between about 1:19 and about 1:300, between about 1:100 and about 1:300, between about 1:100 and about 1000, between about 1:300 and about 1:3333, or between about 1:1000 and 1:3333, inclusive.

In certain embodiments, the solubility of the TZD or pharmaceutically acceptable salt thereof in the vehicle is at least 1 part TZD or pharmaceutically acceptable salt thereof per 3000 parts of vehicle, at least 1 part TZD or pharmaceutically acceptable salt thereof per 1000 parts of vehicle, at least 1 part TZD or pharmaceutically acceptable salt thereof per 500 parts of vehicle, at least 1 part TZD or pharmaceutically acceptable salt thereof per 300 parts of vehicle, or at least 1 part TZD or pharmaceutically acceptable salt thereof per 100 parts of vehicle. In certain embodiments, the solubility of the TZD or pharmaceutically acceptable salt thereof (expressed as part TZD:part vehicle) is between about 1:19 and about 1:3333, between about 1:19 and about 1:100, between about 1:19 and about 1:300, between about 1:100 and about 1:300, between about 1:100 and about 1000, between about 1:300 and about 1:3333, or between about 1:1000 and 1:3333, inclusive.

In some embodiments, the final concentration of the oleic acid is between about 1 percent to about 20 percent by weight, inclusive. In some embodiments, the final concentration of the oleic acid is between about 5 and about 15 percent, about 1 and about 10 percent, about 1 and about 2 percent, about 1 and about 3 percent, about 1 and about 5 percent, about 2 and about 4 percent, about 3 and about 5 percent, about 3 and about 7 percent, about 4 and about 6 percent, about 5 and about 7 percent, about 6 and about 8 percent, about 7 and about 10 percent, about 10 and about 20 percent, about 10 and about 15 percent, or about 15 and about 20 percent, inclusive. In certain embodiments, the final concentration of the oleic acid is about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20% by weight. These percentages are expressed by weight of the total weight of the composition.

In some embodiments, the oleic acid provided in the composition is replaced by another different saturated or unsaturated fatty acid in the composition, e.g., lauric acid, myristic acid, palmitic acid, stearic acid, myrstoleic acid, palmitoleic acid, sapienic acid, linoleic acid, or α-linolenic acid, and in such cases, the above recited embodiments contemplated for oleic acid are also contemplated for the replacement fatty acid. In some embodiments, the fatty acid is unsaturated. In some embodiments, the fatty acid is monounsaturated. In some embodiments, the fatty acid has a chain length of 16 to 20 carbons, inclusive.

In certain embodiments, the composition comprises only one type of monohydroxy alcohol selected from the group consisting of methanol, ethanol, n-propanol, and isopropanol. In some embodiments, the composition comprises two or more different monohydroxy alcohols selected from the group consisting of methanol, ethanol, n-propanol, and isopropanol.

In some embodiments, the final concentration of the one or more monohydroxy alcohols is between about 30 percent and about 99 percent by weight, inclusive. In some embodiments, the final concentration of the one or more monohydroxy alcohols is between about 30 percent and about 40 percent, about 30 percent and about 50 percent, about 40 percent and about 50 percent, about 40 percent and about 60 percent, about 50 percent and about 60 percent, about 50 percent and about 70 percent, about 50 percent and about 80 percent, about 60 percent and about 70 percent, about 60 percent and about 80 percent, about 70 percent and about 80 percent, about 70 percent and about 90 percent, about 80 percent and about 90 percent, about 85 percent and about 95 percent, about 90 percent and about 95 percent, about 90 percent and about 99 percent, and about 95 percent and about 99 percent, inclusive. These percentages are expressed by weight of the total weight of the composition.

In some embodiments, the composition further comprises one or more dihydroxy alcohols, e.g., propylene glycol, 1,3-butanediol, ethylene glycol, or a mixture thereof. In some embodiments, the composition comprises a TZD or pharmaceutically acceptable salt thereof, oleic acid, one or more monohydroxy alcohols, and propylene glycol. In some embodiments, the composition comprises a TZD or pharmaceutically acceptable salt thereof, oleic acid, ethanol, and propylene glycol. In some embodiments, the composition is a solution comprising a TZD or pharmaceutically acceptable salt thereof, oleic acid, ethanol, and propylene glycol. In some embodiments, the composition comprises a TZD or pharmaceutically acceptable salt thereof, oleic acid, isopropanol, and propylene glycol. In some embodiments, the composition is a solution comprising a TZD or pharmaceutically acceptable salt thereof, oleic acid, isopropanol, and propylene glycol. In certain embodiments, the composition consists essentially of the above-recited components.

In some embodiments, the composition comprises rosiglitazone or a pharmaceutically acceptable salt thereof, oleic acid, and a monohydroxy alcohol. In some embodiments, the composition comprises rosiglitazone or a pharmaceutically acceptable salt thereof, oleic acid, and ethanol. In some embodiments, the composition comprises rosiglitazone or a pharmaceutically acceptable salt thereof, oleic acid, and isopropanol. In some embodiments, the composition comprises rosiglitazone or a pharmaceutically acceptable salt thereof, oleic acid, a monohydroxy alcohol, and propylene glycol. In some embodiments, the composition comprises rosiglitazone or a pharmaceutically acceptable salt thereof, oleic acid, ethanol, and propylene glycol. In some embodiments, the composition is a solution comprising rosiglitazone or a pharmaceutically acceptable salt thereof, oleic acid, ethanol, and propylene glycol. In some embodiments, the composition comprises rosiglitazone or a pharmaceutically acceptable salt thereof, oleic acid, isopropanol, and propylene glycol. In some embodiments, the composition is a solution comprising rosiglitazone or a pharmaceutically acceptable salt thereof, oleic acid, isopropanol, and propylene glycol. In certain embodiments, the composition consists essentially of the above-recited components.

In some embodiments, the composition comprises pioglitazone or a pharmaceutically acceptable salt thereof, oleic acid, and a monohydroxy alcohol. In some embodiments, the composition comprises pioglitazone or a pharmaceutically acceptable salt thereof, oleic acid, and ethanol. In some embodiments, the composition comprises pioglitazone or a pharmaceutically acceptable salt thereof, oleic acid, and isopropanol. In some embodiments, the composition comprises pioglitazone or a pharmaceutically acceptable salt thereof, oleic acid, ethanol, and propylene glycol. In some embodiments, the composition is a solution comprising pioglitazone or a pharmaceutically acceptable salt thereof, oleic acid, ethanol, and propylene glycol. In some embodiments, the composition comprises pioglitazone or a pharmaceutically acceptable salt thereof, oleic acid, isopropanol, and propylene glycol. In some embodiments, the composition is a solution comprising pioglitazone or a pharmaceutically acceptable salt thereof, oleic acid, isopropanol, and propylene glycol. In certain embodiments, the composition consists essentially of the above-recited components.

In certain embodiments, the composition comprises only one dihydroxy alcohol selected from the group consisting of propylene glycol, 1,3-butanediol, and ethylene glycol. In some embodiments, the composition comprises two or more dihydroxy alcohols selected from the group consisting of propylene glycol, 1,3-butanediol, and ethylene glycol.

In certain embodiments, the final concentration of the one or more dihydroxy alcohols is between about 5 percent and about 50 percent by weight, inclusive. In some embodiments, the final concentration of the one or more dihydroxy alcohols is between about 5 percent and 10 percent, about 5 percent and about 15 percent, about 10 percent and about 15 percent, about 10 percent and about 20 percent, about 15 percent and about 20 percent, about 15 percent and about 25 percent, about 15 and about 40 percent, about 20 percent and about 25 percent, about 20 percent and 30 percent, about 25 percent and about 30 percent, about 25 percent and about 35 percent, about 30 percent and about 35 percent, about 30 percent and about 40 percent, about 35 percent and about 40 percent, about 35 percent and about 45 percent, about 40 percent and about 50 percent, about 40 percent and about 45 percent, or about 45 percent and about 50 percent, inclusive. In certain embodiments, the composition comprises one or more dihydroxy alcohols in about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30% by weight. These percentages are expressed by weight of the total weight of the composition.

In certain embodiments wherein the dihydroxy alcohol is propylene glycol, the final concentration of the propylene glycol is between about 5 percent and 10 percent, about 5 percent and about 15 percent, about 10 percent and about 15 percent, about 10 percent and about 20 percent, about 15 percent and about 20 percent, about 15 percent and about 25 percent, about 20 percent and about 25 percent, about 20 percent and 30 percent, about 25 percent and about 30 percent, about 25 percent and about 35 percent, about 30 percent and about 35 percent, about 30 percent and about 40 percent, about 35 percent and about 40 percent, about 35 percent and about 45 percent, about 40 percent and about 50 percent, about 40 percent and about 45 percent, or about 45 percent and about 50 percent, inclusive. In certain embodiments, the composition comprises propylene glycol in about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30% by weight. These percentages are expressed by weight of the total weight of the composition.

In some embodiments, the composition comprises water. The water may be, for example, tap water, distilled water, or deionized water. In some embodiments, the final concentration of water is between about 0.1 percent and about 30 percent by weight, inclusive. In some embodiments, the final concentration of water is between about 0.1 percent and 25 percent, about 0.2 percent and about 20 percent, about 0.3 percent and about 15 percent, about 5 and about 10 percent, about 7 and about 10 percent, about 5 and about 15 percent, about 10 and about 15 percent, about 0.4 percent and about 10 percent, about 0.5 percent and about 8 percent, or about 1 percent and about 5 percent, inclusive. These percentages are expressed by weight of the total weight of the composition. In certain embodiments, the water replaces part of the monohydroxy alcohol (e.g., ethanol, isopropanol), which acts as the base component of the composition.

In some embodiments, the composition comprises glycerin (aka glycerol, glycerine). In some embodiments, the final concentration of glycerin is between about 0.1 percent and about 30 percent by weight, inclusive. In some embodiments, the final concentration of glycerin is between about 0.1 percent and 25 percent, about 0.2 percent and about 20 percent, about 0.3 percent and about 15 percent, about 0.4 percent and about 10 percent, about 0.5 percent and about 8 percent, about 1 percent and about 5 percent, inclusive. These percentages are expressed by weight of the total weight of the composition. In certain embodiments, the glycerin replaces part of the monohydroxy alcohol (e.g., ethanol, isopropanol), which acts as the base component of the composition.

In certain embodiments, the composition comprises:

(a) a TZD or pharmaceutically acceptable salt thereof in a concentration of between about 0.3 percent and about 1 percent (by weight), such as about 0.03 and about 0.1 percent, about 0.1 and about 0.3 percent, about 0.1 and about 1 percent, or about 0.3 and about 1 percent (by weight), inclusive;

(b) oleic acid in a concentration of between about 1 percent to about 20 percent by weight, such as between about 5 and about 15 percent, about 1 and about 10 percent, about 1 and about 2 percent, about 1 and about 3 percent, about 1 and about 5 percent, about 2 and about 4 percent, about 3 and about 5 percent, about 3 and about 7 percent, about 4 and about 6 percent, about 5 and about 7 percent, about 6 and about 8 percent, about 7 and about 10 percent, about 10 and about 20 percent, about 10 and about 15 percent, or about 15 and about 20 percent, inclusive, such as about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, or about 20% by weight; and

(c) ethanol or isopropanol in a concentration between about 30 and about 99 percent by weight.

In certain embodiments, the composition comprises:

(a) a TZD or pharmaceutically acceptable salt thereof in a concentration of between about 0.3 percent and about 1 percent (by weight), such as about 0.03 and about 0.1 percent, about 0.1 and about 0.3 percent, about 0.1 and about 1 percent, or about 0.3 and about 1 percent (by weight), inclusive;

(b) oleic acid in a concentration of between about 1 percent to about 20 percent by weight, such as between about 5 and about 15 percent, about 1 and about 10 percent, about 1 and about 2 percent, about 1 and about 3 percent, about 1 and about 5 percent, about 2 and about 4 percent, about 3 and about 5 percent, about 3 and about 7 percent, about 4 and about 6 percent, about 5 and about 7 percent, about 6 and about 8 percent, about 7 and about 10 percent, about 10 and about 20 percent, about 10 and about 15 percent, or about 15 and about 20 percent, inclusive, such as about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, or about 20% by weight; and

(c) ethanol or isopropanol in a concentration of between about 51 percent and 60 percent, 51 percent and about 70 percent, about 60 percent and about 70 percent, about 60 percent and about 80 percent, about 70 percent and about 80 percent, about 70 percent and about 90 percent, about 80 percent and about 90 percent, about 85 percent and about 95 percent, about 90 percent and about 95 percent, about 90 percent and about 99 percent, and about 95 percent and about 99 percent, inclusive.

In certain embodiments, the composition comprises:

(a) a TZD or pharmaceutically acceptable salt thereof in a concentration of between about 0.3 percent and about 1 percent (by weight), such as about 0.03 and about 0.1 percent, about 0.1 and about 0.3 percent, about 0.1 and about 1 percent, or about 0.3 and about 1 percent (by weight), inclusive;

(b) oleic acid in a concentration of between about 1 percent to about 20 percent by weight, such as between about 5 and about 15 percent, about 1 and about 10 percent, about 1 and about 2 percent, about 1 and about 3 percent, about 1 and about 5 percent, about 2 and about 4 percent, about 3 and about 5 percent, about 3 and about 7 percent, about 4 and about 6 percent, about 5 and about 7 percent, about 6 and about 8 percent, about 7 and about 10 percent, about 10 and about 20 percent, about 10 and about 15 percent, or about 15 and about 20 percent, inclusive, such as about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, or about 20% by weight;

(c) ethanol or isopropanol in a concentration of between about 51 percent and 60 percent, 51 percent and about 70 percent, about 60 percent and about 70 percent, about 60 percent and about 80 percent, about 70 percent and about 80 percent, about 70 percent and about 90 percent, about 80 percent and about 90 percent, about 85 percent and about 95 percent, about 90 percent and about 95 percent, about 90 percent and about 99 percent, and about 95 percent and about 99 percent, inclusive; and

(d) another monohydroxy alcohol or dihydroxy alcohol (e.g., propylene glycol) in a final concentration of between about 5 percent and about 50 percent by weight, such as between about 5 percent and 10 percent, about 5 percent and about 15 percent, about 10 percent and about 15 percent, about 10 percent and about 20 percent, about 15 percent and about 20 percent, about 15 percent and about 25 percent, about 20 percent and about 25 percent, about 20 percent and 30 percent, about 25 percent and about 30 percent, about 25 percent and about 35 percent, about 30 percent and about 35 percent, about 30 percent and about 40 percent, about 35 percent and about 40 percent, about 35 percent and about 45 percent, about 40 percent and about 50 percent, about 40 percent and about 45 percent, or about 45 percent and about 50 percent, inclusive, such as about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30% by weight.

In certain embodiments, the composition comprises a TZD or pharmaceutically acceptable salt thereof; oleic acid in about 1% to about 5% by weight, inclusive; ethanol or or isopropanol in about 65% to about 70% by weight, inclusive; and propylene glycol in about 25 to about 30% by weight, inclusive.

In some embodiments, the composition comprises a viscosity-enhancing agent. A viscosity-enhancing agent, as used herein, is a substance that increases the viscosity of a solution or liquid/solid mixture. Exemplary viscosity enhancing agents include, but are not limited to, glycerin; cellulose derivatives (e.g., methylcellulose (MC); hydroxypropylmethylcellulose (HPMC); carboxymethylcellulose (CMC); microcrystalline cellulose (CC); ethyl cellulose; hydroxyethyl cellulose (HEC); hydroxypropyl cellulose (HPC); cellulose); gelatin; starch; hetastarch; poloxamers; pluronics; sodium CMC; sorbitol; acacia; povidone; carbopol; polycarbophil; chitosan; alginate; chitosan glutamate; hyaluronic acid; elastin; hyaluronan; maltodextrin DE; deoxyglycocholate (GDC); polymethacrylic acid; glycols (e.g., polymethylene glycol; polyethylene glycol); cyclodextrins (e.g., sulfobutylether B cyclodextrin); sodium tauro-dihydrofusidate (STDHF); and N-trimethyl chitosan chloride (TMC). In certain embodiments, the viscosity-enhancing agent is a cellulose derivative, e.g., hydroxypropyl cellulose (HPC). In certain embodiments, the composition comprises a viscosity-enhancing agent between about 0.5% and about 5% by weight, inclusive. In certain embodiments, the composition comprises a viscosity enhancing agent in between about 0.5% and about 4%, between about 0.5% and about 3%, between about 0.5% and about 2%, between about 0.5% and about 1%, between about 0.8% and about 5%, between about 0.8% and about 4%, between about 0.8% and about 3%, between about 0.5% and about 2%, or between about 0.5% and about 1%, inclusive. In certain embodiments, the composition comprises a viscosity-enhancing agent in about 0.5%, about 0.6%, about 0.7%, about 0.8%, about 0.9%, about 1%, about 2%, about 3%, about 4%, or about 5% (by weight). These percentages are expressed by weight of the total weight of the composition.

In certain embodiments, the composition may further comprise a buffering agent. The term “buffering agent” refers to a weak acid or base used to maintain the hydrogen ion activity of a solution near a chosen value after the addition of another acid or base. Exemplary buffering agents include a phosphate, acetate, citrate, gluconate, lactate, tartrate, or glutamate.

In certain embodiments, the composition may further comprise other pharmaceutically acceptable excipients including, but not limited to, solvents, diluents or other liquid vehicles, dispersion or suspension aids, surface active agents, isotonic agents, thickening or emulsifying agents, preservatives, lubricants and the like. General considerations in the formulation and/or manufacture of topical compositions can be found, for example, in Remington's Pharmaceutical Sciences, Sixteenth Edition, E. W. Martin (Mack Publishing Co., Easton, Pa., 1980), and Remington: The Science and Practice of Pharmacy, 21^{st} Edition (Lippincott Williams & Wilkins, 2005).

Compositions can be prepared, packaged, and/or sold in bulk, as a single unit dose, and/or as a plurality of single unit doses. As used herein, a “unit dose” is discrete amount of the composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a composition will vary, depending upon the identity, size, and/or condition of the subject treated and further depending upon the route by which the composition is to be administered.

Exemplary preservatives include antioxidants, chelating agents, antimicrobial preservatives, antifungal preservatives, alcohol preservatives, acidic preservatives, and other preservatives.

Exemplary antioxidants include alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal. Chlorobutanol, for example, can be used as a preservative in an ointment formulation at a concentration of 0.001% to 1% by weight (such as 0.5% per weight) of the total weight of the final composition.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant Plus, Phenonip, methylparaben, Germall 115, Germaben II, Neolone, Kathon, and Euxyl. In certain embodiments, the preservative is an antioxidant. In other embodiments, the preservative is a chelating agent.

### Process for Preparing a Pharmaceutical Composition

In another aspect, provided is a process for preparing a composition as described herein, comprising the steps of:

(1) combining oleic acid, an alcohol (e.g., a monohydroxy alcohol, a dihydroxy alcohol, a polyhydroxy alcohol, or mixture thereof) and optionally one or more other compounds to form a first mixture; and

(2) combining a TZD or pharmaceutically acceptable salt thereof in the first mixture to provide a second mixture.

In certain embodiments, the process further comprises the step of (3) combining the dihydroxy alcohol (e.g., propylene glycol) in the second mixture to provide a third mixture. In certain embodiments, the first mixture consists or consistents essentially of an alcohol and oleic acid. In certain embodiments, the first mixture is a solution or gel. In certain embodiments, the second mixture is a solution or a gel. In certain embodiments, the oleic acid dissolves in the alcohol mixture to form the first mixture. In certain embodiments, the TZD or pharmaceutically acceptable salt thereof dissolves in the first mixture to provide the second mixture.

In certain embodiments, the first mixture consists essentially of oleic acid and a monohydroxy alcohol. In certain embodiments, the first mixture consists essentially of oleic acid and ethanol. In certain embodiments, the first mixture consists essentially of oleic acid and methanol. In certain embodiments, the first mixture consists essentially of oleic acid and isopropanol. In certain embodiments, the first mixture consists essentially of oleic acid and n-propanol.

In certain embodiments, the first mixture further comprises a dihydroxy alcohol (e.g., propylene glycol), i.e., to provide a first mixture comprising a oleic acid, a monohydroxy alcohol, and a dihydroxy alcohol. In certain embodiments, the first mixture comprises oleic acid, ethanol, and propylene glycol. In certain embodiments, the first mixture consists essentially of oleic acid, ethanol, and propylene glycol. In certain embodiments, the first mixture comprises oleic acid, isopropanol, and propylene glycol. In certain embodiments, the first mixture consists essentially of oleic acid, isopropanol and propylene glycol.

In certain embodiments, the first mixture consists essentially of oleic acid and a dihydroxy alcohol. In certain embodiments, the first mixture consists essentially of oleic acid and propylene glycol.

In any of the above processes, the aforementioned components (e.g., a thiazolidinedione, oleic acid, monohydroxy alcohol, dihydroxy alcohol) can be combined in different order from that describe above, provided that the process results in a full and uniform dissolution of the thiazolidinedione in the finished product.

For example, in another aspect, provided is a process for preparing a composition as described herein, comprising the steps of:

(1) combining TZD and an alcohol (e.g., a dihydroxy alcohol) and optionally one or more other compounds to form a first mixture; and

(2) combining oleic acid and a monohydroxy alcohol with the first mixture to form a second mixture.

In certain embodiments, any of the mixtures can further comprise an antioxidant, e.g., a form of vitamin E, e.g., alpha-tocopherol.

In certain embodiments, any of the mixtures can further comprise a buffering agent.

In certain embodiments, any of the mixtures can further comprise a viscosity-enhancing agent, i.e., any of the above processes further comprises a step of adding a viscosity-enhancing agent to the first, second, or third mixture.

In certain embodiments, the process comprises combining ethanol, oleic acid, a TZD or pharmaceutically acceptable salt thereof, and optionally one or more other excipients, e.g., an antioxidant, buffering agent, and/or viscosity-enhancing agent. In certain embodiments, the process comprises combining isopropanol, oleic acid, a TZD or pharmaceutically acceptable salt thereof, and optionally one or more other excipients, e.g., an antioxidant, buffering agent, and/or viscosity-enhancing agent.

In certain embodiments, the relative amount of the alcohol (e.g., a monohydroxy alcohol and/or dihydroxy alcohol), oleic acid, and TZD or pharmaceutically acceptable salt thereof are as described herein.

In certain embodiments, the solubility of the TZD or pharmaceutically acceptable salt thereof is at least 1 part TZD or pharmaceutically acceptable salt thereof per 3000 parts of vehicle, at least 1 part TZD or pharmaceutically acceptable salt thereof per 1000 parts of vehicle, at least 1 part TZD or pharmaceutically acceptable salt thereof per 500 parts of vehicle, at least 1 part TZD or pharmaceutically acceptable salt thereof per 300 parts of vehicle, or at least 1 part TZD or pharmaceutically acceptable salt thereof per 100 parts of vehicle. As expressed in this paragraph, the solubility is measured at 25° C.

In certain embodiments, the solubility of the TZD or pharmaceutically acceptable salt thereof in the vehicle is at least 0.03%, at least 0.05%, at least 0.1%, at least 0.2%, at least 0.3%, at least 0.5%, at least 0.7%, or at least 1%, inclusive, w/w or w/v. The percentages in this paragraph are expressed as the weight of TZD relative to the total weight or total volume of the vehicle. As expressed in this paragraph, the solubility is measured at 25° C.

### Methods of Treatment

As generally described herein, provided is a method for treating a medical condition or ameliorating a cosmetic condition in a body of a subject in need thereof, the method comprising applying to the skin of the body one or more of the compositions described herein. In certain embodiments, the medical or cosmetic condition is a deficiency of adipose tissue. The deficiency can affect the body locally or diffusely. The adipose tissue affected by the deficiency can be subcutaneous adipose tissue.

The composition can be applied to the skin three times a day, two times a day, once a day, every other day, every third day, every week, every two weeks, every three weeks, or every four weeks. In certain embodiments, the composition can be applied using multiple administrations (e.g., two, three, four, five, six, seven, eight, nine, ten, eleven, twelve, thirteen, fourteen, or more administrations).

A therapeutically effective amount of the active ingredient for administration one or more times a day to may comprise about 0.1 mg to about 1 mg per cm² of skin, about 0.1 mg to about 10 mg per cm² of skin, about 0.1 mg to about 100 mg per cm² of skin, about 1 mg to about 10 mg per cm² of skin, about 1 mg to about 100 mg per cm² of skin, or about 10 mg to about 100 mg per cm² of skin.

The area of skin to which the composition is applied at any one time may comprise about 1 cm² to about 10,000 cm², about 1 cm² to about 1,000 cm², about 1 cm² to about 100 cm², about 1 cm² to about 10 cm², about 10 cm² to about 100 cm², about 10 cm² to about 1,000 cm², about 100 cm² to about 1,000 cm², or about 1,000 cm² to about 10,000 cm², inclusive.

In certain embodiments where the subject has a deficiency of subcutaneous fat, the method increases fat locally at the part of the body where the composition is applied (e.g., at least a 10%, at least a 20%, at least a 30%, or at least a 40% increase in subcutaneous fat thickness) compared to a control site on the subject, or compared to the baseline (pre-administration) subcutaneous fat measurement, e.g., between 10% to 100%, increase, inclusive. In certain embodiments, the increase is at least 10% greater at the treatment site than at a control site or compared to the baseline measurement. In certain embodiments, the increase is at least 20% greater at the treatment site than at a control site or compared to the baseline measurement. In certain embodiments, the increase is at least 30% greater at the treatment site than at a control site or compared to the baseline measurement. In certain embodiments, the increase is at least 40% greater at the treatment site than at a control site or compared to the baseline measurement.

In some embodiments, the subject suffers from a subcutaneous fat deficiency, and the method is directed to treating the subcutaneous fat delivery. In some embodiments, the subcutaneous fat deficiency is associated with a metabolic disorder. Exemplary metabolic disorders associated with subcutaneous fat deficiency include, but are not limited to, insulin resistance, diabetes (e.g., lipoatrophic diabetes), lipase deficiency, wasting, malnutrition, paraneoplastic condition, anorexia, pernicious anemia, celiac disease, and malabsorption syndrome.

In some embodiments, the subcutaneous fat deficiency is associated with an inflammatory condition. Exemplary inflammatory conditions associated with subcutaneous fat deficiency include, but are not limited to, complement component 3 (C3) deficiency, membranoproliferative glomerulonephritis, systemic lupus erythematosus, dermatomyositis, rheumatoid arthritis, temporal arteritis, and leukocytoclastic vasculitis.

In some embodiments, the subcutaneous fat deficiency is acquired. As used herein, “acquired” means a disorder that is not congenital. Exemplary conditions associated with acquired subcutaneous fat deficiency include, but are not limited to, HIV-associated lipodystrophy, lipidema, acquired partial lipodystrophy (Barraquer-Simons syndrome), acquired generalized lipodystrophy, Parry-Romberg syndrome, juvenile dermatomyositis, centrifugal abdominal lipodystrophy (lipodystrophia centrifugalis abdominalis infantilis), lipoatrophia annularis (Ferreira-Marques lipoatrophia), and localized lipodystrophy.

In some embodiments, the subcutaneous fat deficiency is congenital. Exemplary congenital conditions associated with subcutaneous fat deficiency include, but are not limited to, congenital generalized lipodystrophy (Beradinelli-Seip syndrome), familial partial dystrophy (e.g., Kobberling-type, Dunnigan type, or Type 3), Nakajo-Nishimura syndrome, Cockayne syndrome, SHORT syndrome, AREDYLD syndrome, mandibuloacral dysplasia, Keppen-Lubinsky syndrome, POEMS syndrome, Werner syndrome, Hutchinson-Gilford syndrome, and progeria. In some embodiments, the subcutaneous fat deficiency is caused by a lipoatrophy-causing mutation in a gene selected from the group consisting of APLD, AKT2, C3, CAV1, CGL1 (AGPAT2), and CGL2 (BSCL2), LMF1, LMNA, PLIN1, PPARG, PSMB8, PTRF, and ZMPSTE24.

In some embodiments, the subcutaneous fat deficiency is caused by a medication. Exemplary medications known to cause subcutaneous fat deficiency include, but are not limited to, an antiretroviral (see, e.g., Domingo et al., AIDS Rev 2012;14:112-123), an antibiotic (see, e.g., Kayikcioglu et al., J Pediatr 1996;129:166-167), iron, a growth hormone, a fat solubilizer (see, e.g., U.S. Pat. No. 7,622,130), and a corticosteroid and/or a beta-adrenergic agonist (see, e.g., U.S. patent application Ser. No. 13/204,423). Exemplary antiretroviral medications are non-nucleoside reverse transcriptase inhibitors (e.g., efavirenz), nucleoside/nucleotide analog reverse transcriptase inhibitors (e.g., zidovudine), and HIV-1 protease inhibitors (e.g., nelfinavir). Exemplary corticosteroids include fluticasone, triamcinolone, betamethasone, prednisolone, methylprednisolone, and dexamethasone. Exemplary antibiotics include penicillin. An exemplary beta-adrenergic agonists is salmeterol. An exemplary fat solubilizer is deoxycholate.

In some embodiments, the subcutaneous fat deficiency is caused by surgery. For example, thiazolidinediones and/or an orexigenic compounds, as described herein, may also be useful as an adjunct to any of various kinds of surgery, whether used in the pre-operative, peri-operative, or post-operative period. In some embodiments, a loss or deficiency of fat is caused by an injury. In some embodiments, the injury is selected from the group consisting of mechanical injury, burn, cryoinjury, and radiation injury.

In some embodiments, the subject does not necessarily suffer from a deficiency of fat. For example, in some embodiments, the subject suffers from wrinkles of the skin, e.g., the skin is affected by wrinkles, and the method is directed to ameliorating the wrinkles, e.g., by reducing the depth or visibility of the wrinkles

In certain embodiments, the skin is on the face, forehead, periorbital region of the face, midface, cheeks, chin, lips, breast, limbs, hands, trunk, hips, or buttocks.

In other embodiments, the subject suffers from dissatisfaction with the size or contour of a body part, and the method is directed to modifying the contour of the body part. As used herein, “modifying the contour of a body part” refers to changing the shape of the body part, for example, by augmenting the whole body part, by augmenting one or more area of the body part, or by augmenting one or more areas neighboring the body part. For example, the contour of the cheeks could be modified by augmenting the cheeks as a whole, by selectively augmenting only a portion of the cheeks (e.g., the malar eminences). Selective augmentation of a particular area is obtained by selectively treating the particular area, as described herein. Exemplary body parts contemplated for modification include, for example, the head (e.g., face such as the forehead, forehead, periorbital region, cheeks, chin, lips, and other anterior structures from top of forehead to bottom of chin), breast, limbs, hands, trunk, hips, and buttocks.

### EXAMPLES

In light of the foregoing description, the specific non-limiting examples presented below are for illustrative purposes and not intended to limit the scope of the invention in any way.

### Example 1

The solubility of rosiglitazone maleate and pioglitazone hydrochloride in various solvents was tested by a standard method, i.e., serial addition of solvent to a known mass of the TZD compound, until full dissolution was observed. Additional solubility results were obtained from the literature. Table 1A shows the collated results. The results indicate that addition of oleic acid 3% w/w to ethanol-based vehicles substantially increased the solubility of two representative TZD compounds in those vehicles (compare Experiment 3 to Experiments 4 and 6 for rosiglitazone maleate, and compare Experiment 2 to Experiments 4 and 5 for pioglitazone hydrochloride). This phenomenon occurred in formulations comprising water, as well as formulations lacking water. The results further indicate that addition of oleic acid 3% w/w to an isopropanol-based vehicle substantially increased the solubility of rosiglitazone maleate in that vehicle (compare Experiment 8 to Experiment 7). Next, the solubility of troglitazone in various solvents was tested by a standard method, i.e., serial addition of solvent to a known mass of troglitazone, until full dissolution was observed. Additional solubility results were obtained from the literature. Table 1B shows the collated results. The results indicate that addition of oleic acid 3% w/w to an ethanol-based vehicle substantially increased the solubility of troglitazone in that vehicle (compare Experiment 11 to Experiment 12).

TABLE 1A

| | | Solubility at 25° C. | | | |
|---|---|---|---|---|---|
| | | rosiglitazone | | pioglitazone | |
| | Solvent | maleate | | hydrochloride | |
| 1 | Water | NP | | 0.05 | mg/mL^{b} |
| 2 | Ethanol | 0.25 | mg/mL^{a} | 4 | mg/mL^{c} |
| 3 | 70% ethanol, 30% propylene glycol | 0.13 | mg/g* | NP | |
| | (PG) | | | | |
| 4 | 70% ethanol, 27% PG, 3% oleic acid | ~10 | mg/g* | ~9 | mg/g* |

| | | | | |
|---|---|---|---|---|
| 5 | 65% ethanol, 25% PG, 7% water, | NP | ~9 | mg/g* |
| | 3% oleic acid | | | |

| | | | | |
|---|---|---|---|---|
| 6 | 63% ethanol, 24% PG, 10% water, | ~10 | mg/g* | NP |
| | 3% oleic acid | | | |
| 7 | 70% isopropyl alcohol, 30% PG | 0.4 | mg/g* | NP |
| 8 | 70% isopropyl alcohol, 27% PG, | 1.2 | mg/g* | NP |
| | 3% oleic acid | | | |

*Tested by inventor(s)
NP = Not performed
^{a}A Cayman Chemical, accessed May 15, 2014: www.caymanchem.com/pdfs/11884.pdf
^{b}Jahnavi et al, Indian J Pharm. Edu. Res. 2013; 47: 15
^{c}Santa Cruz Biotechnology, accessed May 15, 2014: www.scbt.com/datasheet-204848-pioglitazone-hydrochloride.html

TABLE 1B

| | | Solubility at 25° C. |
|---|---|---|
| | Solvent | troglitazone |
| 9 | Water | 0.02 mg/mL^{a} |
| 10 | Ethanol | 0.3 mg/mL^{b} |
| 11 | 70% ethanol, 30% propylene glycol (PG) | 0.3 mg/g* |
| 12 | 70% ethanol, 27% PG, 3% oleic acid | 0.6 mg/g* |

*Tested by inventor(s)
NP = Not performed
^{a}Young et al, Br J Clin Pharmacol 1998; 45: 31-35.
^{b}A Cayman Chemical, accessed Nov. 15, 2014: www.caymanchem.com/pdfs/71750.pdf

### Example 2

A cosmetic composition was prepared as follows:

TABLE 2

| Ingredients per 100 g of final product | | Amount | |
|---|---|---|---|
| TZD | Rosiglitazone maleate | 0.5 | g |
| Antioxidant | alpha-tocopherol | 0.002 | g |
| Percutaneous carrier | Ethanol, anhydrous | 68.5 | g |
| | Propylene glycol | 27 | g |
| | Oleic acid | 3 | g |
| Viscosity enhancing agent | Hydroxypropylcellulose | 2 | g |
| | (e.g., Klucel ® Grade HF) | | |

The alpha-tocopherol was added to the ethanol, and the solution was mixed. Propylene glycol and oleic acid were added, and the resulting solution was mixed. The rosiglitazone maleate was added, and the resulting solution was mixed. The hydroxypropylcellulose was added and thoroughly mixed to yield a gel with a final rosiglitazone concentration of 0.5% (w/w).

Similar products were made comprising 0.1% and 1% (w/w) rosiglitazone maleate, with ethanol content accounting for the difference.

The compositions comprising 0.1% and 0.5% rosiglitazone maleate were stored at room temperature for about 6 weeks, then evaluated for appearance and chemical stability by High Performance Liquid Chromatography (HPLC). The appearance and rosiglitazone concentrations were stable; no degradants were detected.

### Example 3

Skin permeation studies were conducted with various formulations of rosiglitazone, ex vivo, on fresh minipig skin. Harvested skin was mounted on a standard (Franz-type) diffusion cell apparatus. All test articles contained 0.1% (weight/weight) of rosiglitazone. Each test article (8 mg) was uniformly applied to a skin surface of 0.8 cm². Treated skin was left open to the atmosphere to simulate clinical conditions. Receptor fluid flowed continuously over 24 hours and was collected in fractions. The amount of rosiglitazone in these fractions was determined by Liquid Chromatography/Tandem Mass Spectrometry. The following amounts of drug were recovered from receptor fluid over 24 hours:

TABLE 3

| Formulation | Drug mass (ng, mean) |
|---|---|
| Ethanol 70%, PG 30% | 770 |
| Ethanol 70%, PG 27%, oleic acid 3% | 3129 |

PG = propylene glycol

### Example 4

A prospective controlled trial was performed in Gottingen minipigs (n=4). On one flank of each pig, a 50 cm² area was treated once daily with a composition according to Example 2 comprising 1% rosiglitazone (as maleate salt). On the opposite flank of each pig, a 50 cm² area was treated once daily with the corresponding vehicle. After 42 days, necropsy was done and samples of skin, subcutaneous fat, and muscle were collected en bloc from each treatment area in a controlled fashion. Samples were examined microscopically and measured digitally. The treatment was well tolerated, with no evidence of skin irritation or other clinical observations. Treatment with percutaneous rosiglitazone maleate was associated with an increase in subcutaneous fat thickness of about 48% (p=0.0002), as compared to contralateral tissue treated with placebo.

### Other Embodiments

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

What is claimed is:

1. A composition comprising:
a. a thiazolidinedione selected from the group consisting of rosiglitazone, pioglitazone, troglitazone, ciglitazone, netoglitazone, rivoglitazone, and pharmaceutically acceptable salts thereof; and
b. a vehicle comprising oleic acid and a monohydroxy alcohol.

2. The composition of claim 1, wherein the monohydroxy alcohol is selected from the group consisting of methanol, ethanol, isopropanol, and n-propanol.

3. The composition of claim 2, wherein the monohydroxy alcohol is ethanol.

4. The composition of claim 2, wherein the monohydroxy alcohol is isopropanol.

5. The composition of claim 1, wherein the solubility of the thiazolidinedione in the vehicle is at least 1 part thiazolidinedione or pharmaceutically acceptable salt thereof per 3000 parts of vehicle.

6. The composition of claim 5, wherein the solubility of the thiazolidinedione in the vehicle is at least 1 part thiazolidinedione or pharmaceutically acceptable salt thereof per 1000 parts of vehicle.

7. The composition of claim 6, wherein the solubility of the thiazolidinedione in the vehicle is at least 1 part thiazolidinedione or pharmaceutically acceptable salt thereof per 300 parts of vehicle.

8. The composition of claim 7, wherein the solubility of the thiazolidinedione in the vehicle is at least 1 part thiazolidinedione or pharmaceutically acceptable salt thereof per 100 parts of vehicle.

9. The composition of claim 1, wherein the concentration of the thiazolidinedione is between about 0.03% and about 1% of the total weight of the composition.

10. The composition of claim 1, wherein the thiazolidinedione is rosiglitazone or a pharmaceutically acceptable salt thereof.

11. The composition of claim 10, wherein the thiazolidinedione is rosiglitazone maleate.

12. The composition of claim 1, wherein the thiazolidinedione is pioglitazone or a pharmaceutically acceptable salt thereof.

13. The composition of claim 10, wherein the thiazolidinedione is pioglitazone hydrochloride.

14. The composition of claim 1, wherein the concentration of the oleic acid is between about 1% and about 5% of the total weight of the composition.

15. The composition of claim 1, wherein the concentration of the monohydroxy alcohol is between about 50% and about 80% of the total weight of the composition.

16. The composition of claim 1, further comprising propylene glycol.

17. The composition of claim 16, wherein the concentration of the propylene glycol is between about 20% and about 30% of the total weight of the composition.

18. The composition of claim 1, further comprising glycerin.

19. The composition of claim 1, further comprising water.

20. A process for preparing a topical composition, comprising the steps of: (1) combining oleic acid and a monohydroxy alcohol and optionally one or more other compounds to form a first mixture; and (2) combining a TZD or pharmaceutically acceptable salt thereof in the first mixture to provide a second mixture.

21. The process of claim 20, further comprising the step of adding a viscosity-enhancing agent to the mixture.

22. The process of claim 20, wherein the monohydroxy alcohol is selected from the group consisting of methanol, ethanol, isopropanol, and n-propanol.

23. The process of claim 22, wherein the monohydroxy alcohol is ethanol.

24. The process of claim 22, wherein the monohydroxy alcohol is isopropanol.
